(19) United States
(12) Patent Application Publication
(10) Pub. No.: US 2010/0324475 A1
(43) Pub. Date: Dec. 23, 2010

(54) POLYMER RELEASING ORTHOPEDIC TREATMENT SYSTEM

(76) Inventor: James K. Brannon, Leawood, KS (US)

Correspondence Address: INTELLECTUAL PROPERTY CENTER, LLC, 9233 WARD PARKWAY, SUITE 100, KANSAS CITY, MO, 64114, US

(21) Appl. No.: 12/820,133

(22) Filed: Jun. 21, 2010

Related U.S. Application Data
(60) Provisional application No. 61/218,757, filed on Jun. 19, 2009.

(51) Int. Cl. A61M 5/178; A61M 1/00

(52) U.S. Cl. 604/36; 604/28

(57) ABSTRACT

The present invention provides an orthopedic treatment system and a method for selectively releasing a growth agent to a surgical site associated with a bone structure that was borne by endoscopically debrided osseus tissue and adapted for receiving the growth agent.

### CROSS-REFERENCE TO RELATED APPLICATION

This application claims the benefit of the prior filed U.S. provisional application No. 61/218,757 filed Jun. 19, 2009 which is incorporated herein by reference.

### FIELD OF THE INVENTION

The present invention relates to medical devices and more particularly to a system for delivery of polymer growth agents to a surgical site for treating bone surfaces within the body and providing support thereof.

### BACKGROUND OF THE INVENTION

Orthopedic bone is subject to a variety of diseases and conditions which cause pain and disability including, but not limited to, voids, fractures, defects, Osteonecrosis, Bone Spurs, Bone Tumor, Enchondroma, Extra-Abdominal Desmoid Tumors, Fibrous Dysplasia, Giant Cell Tumor of Bone, Infectious Arthritis, Osteomyelitis, Klippel-Feil Syndrome, Limb Length Discrepancy, Osteochondritis Dissecans, Osteochondroma, Osteomalacia, Osteomyelitis, Osteopetroses, Proteus Syndrome, Renal Osteodystrophy and Unicameral Bone Cyst. Additionally, the connecting tissues are subject to injuries, including fractures and torn ligaments.

The pain and disability related to these injuries, conditions and diseases often results in impaired movement and decreased health. In addition, these injuries may last for a long time including months and years and may lead to loss of mobility. In addition, under normal conditions, the bone repair process may not heal properly. For example, if the bone mends itself, a weakened bone structure may result even though the bone is healed. In addition, infection may occur and delay the healing process.

A variety of techniques have been used to treat these injuries, conditions and diseases to return the bone segment to its normal healthy condition, however, these techniques have disadvantages. Additionally, some techniques have been used to expedite the healing process; however, these techniques also have disadvantages.

One method of treatment is related rods and plates to secure the bone segments together; however, this process requires further damage to the bone does not heal the diseased bone segment itself. In addition, this procedure precludes the use of this process on children, as the destruction of the inner lining disrupts or inhibits growth. Another method is related to injecting bone cement into surgically prepared bone cavities, however, this treatment method requires different injections under different conditions and these injections have limited duration and are only generally effective at the time of the injection or for a short time thereafter.

Therefore there exists a need for an orthopedic fixation device which provides structural stability to the bone segment while providing for the controlled release of a polymer for healing and strengthening the bone segment.

### SUMMARY OF THE INVENTION

The present invention is directed to a new and improved system and method for delivery of a growth agent to a surgical site. A polymer releasing orthopedic treatment system for selectively delivering a growth agent to a surgical site associated with a bone structure that was borne by endoscopically debrided osseus tissue and adapted for receiving the growth agent, the polymer releasing orthopedic treatment system comprising an endoscope having a endoscopic passageway extending between an endoscopic connector and the surgical site, an inflatable orthopedic device in communication with a growth agent delivery system, adapted for delivery of said growth agent to said surgical site through said endoscopic connector, said endoscopic passage adapted for receipt of the inflatable orthopedic device, said inflatable orthopedic device having a receiving end and an outer membrane, said receiving end receiving said growth agent for inflation of said outer membrane, and said outer membrane having at least one dispersing site located along said outer membrane for selective dispersement of said growth agent to said bone structure.

### BRIEF DESCRIPTION OF THE DRAWINGS

FIG. 1 is an exemplary embodiment of a growth agent delivery system, an endoscopic passageway, and an inflatable orthopedic device at a surgical site.

FIG. 2 illustrates an exemplary embodiment of a stabilizer and an inflatable orthopedic device at a surgical site.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, (but merely as a basis for the claims) and as a representative basis for teaching one skilled in the art to variously employ the present invention in virtually any appropriately detailed structure.

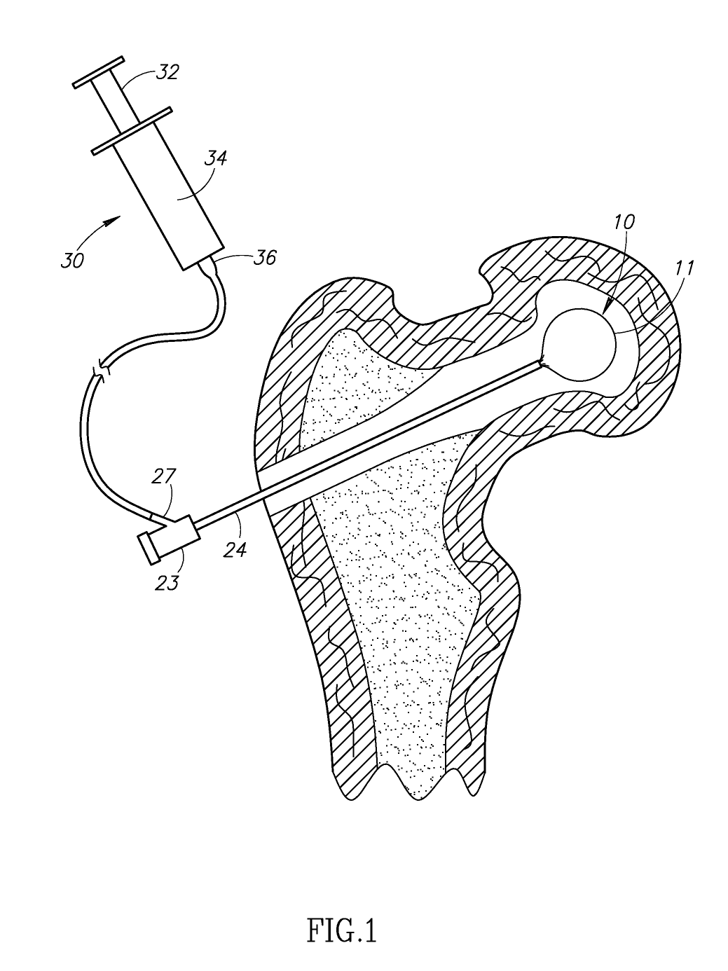

FIG. 1 illustrates an exemplary embodiment of a device for delivery of bone growth agents and bearing at least part of the load of debrided osseus tissue as it may exist at a surgical site. The embodiment is comprised of an inflatable orthopedic device 10, a stabilizer 40, an endoscopic passage 24, and an actuator 32.

The preferred substance to be delivered into the inflatable orthopedic device 10 is a growth agent to induce or aid in bone formation, healing, and repair. The growth agents are preferably in fluid form, such as a liquid or gel but may also be in a powder or other form. Such growth agents may include stem cells, bone marrow, antibiotics, bone morphogenic protein, methylmethylacrylate cement, or other substances known in the art. However, other substances may be delivered to the surgical site with the system. Where growth agent is used in this application, it is to be understood that another substance may be substituted in its place.

The growth agent delivery system 30 is employed to deliver the growth agent to the inflatable orthopedic device 10. The growth agent delivery system 30 includes a dispenser 33 in reciprocal receipt of the actuator 32, a growth agent supply 34, and a dispensing valve 36. The growth agent supply 34 contains a predetermined amount of growth agent to be dispensed. Pressure from the actuator 32 may facilitate transmission of the growth agent from the dispenser 33 along the endoscopic passage 24 into the inflatable orthopedic device 10. A plunger coupled to a tube in communication with the endoscope 20 may be one version of the growth agent delivery system 30. Additional data on growth agents for bone cement is contained in U.S. Pat. No. 4,576,152, which is incorporated by reference.

The growth agent delivery system 30 may be connectably secured to a receiving end of the endoscopic connector 23 associated with the endoscope 20, spaced apart from the endoscopic channel 25. This configuration may provide a passageway for the growth agent to travel to the inflatable device 10 for dispersement at the surgical site. In an alternative configuration, the dispensing valve 36 may be directly coupled to the endoscopic connector 23.

Another aspect of the present invention, the endoscope 20 in communication with the growth agent delivery system 30, is illustrated with an endoscopic valve 27 extending outwardly from an endoscopic connector 23 and in communication with an endoscopic passage 24. The endoscopic connector 23 extends proximally from the endoscopic passage 24 and facilitates transmission of solids, fluids or semi-solids along the endoscopic passage 24. A channel (not shown) may be provided which extends internally from the endoscopic connector 23, outwardly, between the connector and the endoscopic valve 27. The endoscopic valve 27 in operation, may be engaged to restrict flow between the growth agent delivery system 30 and the endoscopic passage 24, through for example rotation of the endoscopic valve 27. In addition, the actuator 32 may be used to regulate the flow of the growth agent to the surgical site through the endoscopic passage 24. The endoscopic valve 27 is adapted for communication with the growth agent delivery system 30 via the dispensing valve 36 with for example, and not as a limitation, a flexible tube, pipe or other conduit extending between the endoscopic valve 27 and the dispensing valve 36.

The endoscopic passage 24, in one embodiment, is composed of a flexible biocompatible material, which is sufficiently rigid to permit material such as the inflatable orthopedic device 10, a needle, or transport instrument 22 to traverse along the interior of the passage 24. The endoscopic passage 24 is preferably a single channel, cylindrically shaped structure adapted to receive fluid or solid material and appropriately dimensioned therefore. The passageway 24 has an inner cylindrical surface adapted for transmitting the received material traversely through the interior endoscopic passage 24 to the surgical site. The transmitted material may include, solid, liquid or semi-solid materials adapted for inflation and growth at the surgical site. The endoscopic connector 23 provides access to the endoscopic passage 24. When desired, a transport instrument 22, such as a plunger or a removable elongated rod or wire with a shaped tip, may be utilized for transmitting the received material through the endoscopic passage 24 to the surgical site. Alternatively, the growth agent may be received by the flexible inflatable device for transmission along the interior endoscopic channel 25, to the surgical site. Additional and/or alternatively, a lubricant may be used to reduce any undesired friction. Where it is desired, the actuator 32, in communication with the endoscopic passage 24, may provide pressure for use in transmitting the growth agent, the inflatable orthopedic device or for inflating the inflatable orthopedic device 10 in receipt of the growth agent.

In one illustrated embodiment, the dispensing valve 36 extends from the dispenser 33 in receipt of the growth agent. The growth agent is selectively transmitted through the dispensing valve 36 as the actuator 32 is telescopically received by the dispenser 33. The growth agent travels along the conduit extending from the dispensing valve to the endoscopic valve 27, where the growth agent is selectively passed along the endoscopic passageway 24. The inflatable orthopedic device may be positioned within the endoscopic passageway 24 with an end adapted for receiving the transmitted growth agent at one end of the inflatable orthopedic device. The opposite end of the orthopedic device is adapted for inflation with the received growth agent, at a distal end of the endoscopic passageway.

The surgical site is associated with bone structure adapted for bone growth activity, such as, but not limited to, bone formation, healing, and repair or where the growth agent is desirably dispensed. In another embodiment, the inflatable orthopedic device 10 is releasably coupled to one end of the endoscopic passage 24 in a deflated state. In operation, the endoscopic passage 24 is passed subcutaneously through the incision to the surgical site. The inflatable orthopedic device 10 is transmitted along the endoscopic channel 25 to the distal end of the endoscopic passage 24. At the distal end of the inflatable orthopedic device 10 receives the growth agent, delivered by the growth agent delivery system 30, the inflatable orthopedic device 10 extending radially from the distal end with the received growth agent.

Depending on the delivery of the growth agent, an outer membrane 11 of the inflatable orthopedic device 10 may extend to the bone structure adapted for bone growth activity, whereupon the inflatable orthopedic device may support the bone structure. A seal (not shown) such as but not limited to a flap seal, may be located at the distal end of the endoscopic passageway 24 for position between the endoscopic passageway 24 and the flexible orthopedic device 10. In addition, at least one dispensing site 12, associated with the outer membrane 11, may extend towards the bone structure, the dispensing site 12 being adapted for dispensing the growth agent at the outer membrane 11. The outer membrane 11 is preferably fabricated from a material which is flexible, has high tensile strength, and is biocompatible. In some versions of this embodiment, a suitable material should be a non-endothelial material or other materials known in the art which may be self-sealing for resealing the dispensing site(s) 12 associated with the outer membrane 11 so that upon puncture of the outer membrane 11 by a needle, bone or other object, the membrane 11 is resealed and undesired release of growth agent from the puncture site is prevented. For a discussion of bone generation, see applicant's U.S Pat. No. 6,872,184, the contents of which are incorporated herein.

In operation, the inflatable orthopedic device 10 is positioned with the outer membrane 11 positioned towards the distal end of the endoscopic passage 24 and the receiving end towards the proximal end of the endoscopic passage 24. As the growth agent is dispersed from the growth agent delivery system 10, it at least partially occupies the inflatable orthopedic device 10, providing movement of the inflatable orthopedic device through the endoscopic passage 24 towards the bone structure. As the growth agent is dispensed at the bone structure, surrounding platelettes may adhere to the outer membrane 11 limiting the release of the growth agent at the surgical site. The outer membrane 11 may continue to inflate or deflate depending in part upon the parameters of the outer membrane 11 as additional growth agent is dispensed to the surgical site by the growth agent delivery system. Over time, the outer membrane 11 may include a material adapted for absorption within the bone structure, such as the bioresborable material disclosed in U.S. Pat. No. 6,280,473, the contents of which are incorporated by reference. Additionally, as the outer membrane 11 is absorbed, additional growth agents may be dispensed at the surgical site for the surrounding bone structure. The dispensing site(s) 12 may vary in size, but may have an average dimension of approximately 200 μm, but with the adherence of the platellettes may decrease in effective size to less than 25 μm.

Optionally, the outer membrane 11 may be coated with a substance to protect the membrane 11 from puncture and provide additional support for the bone structure. In a preferred embodiment, the outer membrane 11 may adjoin the bone structure providing support and preventing further emancipation of any surrounding bone structure during bone growth. In this way, the growth agent may be selected which has reduced compaction properties allowing for improved stability and support of the bone structure. Some potential outer membrane fabricants are disclosed in U.S. Pat. App. No. 20080300684, which is hereby incorporated by reference.

The dimensions of the inflatable orthopedic device 10 vary according the surgical event. For example, a spherical inflatable orthopedic device 10 may have a diameter ranging from less than one centimeter to several centimeters. Likewise, the thickness of the membrane 11 varies with the surgical event based on factors such as the growth agent type and pressure, load bearing needs, and presence of acutely angled bone.

The seal 13 may be used to selectively control the introduction of the growth agent and to prevent undesired release from the inflatable orthopedic device 10, the distal end of the endoscopic passage 24 or both. In operation, the seal 13 may extend from the proximal end of endoscopic passage 24 or the receiving end of the inflatable orthopedic device 10 for disrupting fluid communication between the endoscopic passage 24 and the surgical site. The seal 13 may be further controlled for dispensing additional growth agent to the inflatable orthopedic device 10. When the seal 13 is closed, the dispensing of the growth agent is controlled by the dispensing sites 12 associated with the outer membrane 11.

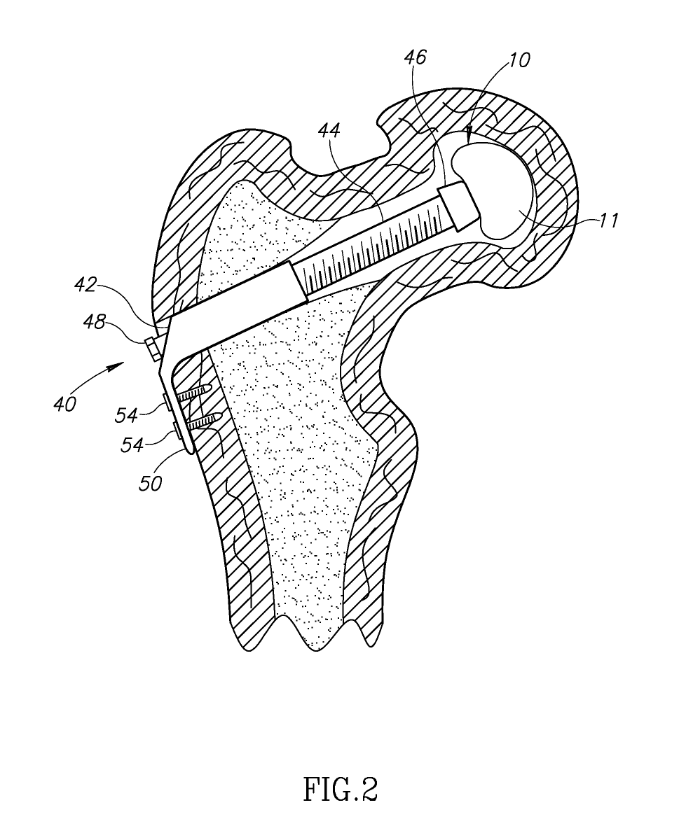

Preferably, the inflatable orthopedic device 10 is delivered to the surgical site and located where it supports at least a portion of the bone structure that was borne by the debrided osseus tissue. Discussion of alternate support structures, such as cross-linking multiple support structures, is provided in U.S. Pat. No. 6,749,614, U.S. Pat. No. 5,658,310, U.S. Pat. No. 5,423,850, U.S. Pat. No. 5,102,413 and U.S. Pat. App. No. 20040193270, which are incorporated by reference. As the inflatable orthopedic device extends radially towards the bone structure, the outer membrane may be positioned adjacent to a first bone surface as illustrated in FIG. 2. As depicted, the outer membrane 11 is radially extended until a sufficient portion of the outer membrane 11 adjoins the surrounding bone structure in such a way that the outer member 11 at least partially deforms in accordance with the surrounding bone structure. The selected growth agent and outer membrane may accordingly be selected to provide desired support of the surrounding bone structure.

The inflatable orthopedic device 10 can be of regularly or irregularly shaped. Key factors in selecting a desirable shape include influencing and controlling the potential load distribution, as well as the necessary locations for dispensing growth agent. The inflatable orthopedic device 10 can include shapes such as spherical, cylindrical, kidney, or doughnuts. Discussion of implant shapes is disclosed in U.S. Pat. Nos. 6,066,154 and 5,827,289, which are hereby incorporated by reference. The inflatable orthopedic device 10 can be prefabricated in the desired shapes. Alternatively, the inflatable orthopedic device's 10 shape and dimensions may be customized and optimized for an individual patient using factors such as the location of the surgical site, the characteristics of the surgical site, the size of the surrounding bone structure, and the load which must be borne at the surgical site. As desired the inflatable orthopedic device 10 may be shaped and dimensioned to maximally bear the load of the surrounding bone structure. Alternatively, the shape of the inflatable orthopedic device may be configured for optimal disbursement of the growth agent to the surgical site.

As the inflatable orthopedic device receives the growth agent within the surgical site, the growth agent is passed through at least one dispensing site 12 associated with the outer membrane to the surrounding bone structure associated with the surgical site. As depicted, the dispensing site 12 may be a passage through the outer membrane 11 for release of the growth agent into the surgical site. The dispensing site 12 is configured according to the desired release characteristics and growth agent properties, with multiple dispensing sites, larger dispensing sites, or shaped according to the desired effect, such as but not limited to the desired flow rate, duration, distance or pressure. For example, where a higher release rate or more viscous growth agent is utilized, the dispensing site 12 may have a larger dimension. The number of dispensing sites 12 on the membrane 11 varies according to the surrounding bone structure and associated release locations.

The inflatable orthopedic device 10 is passed subcutaneously into the surgical site, preferably through the endoscopic passage 24. After receiving the inflatable orthopedic device 10 within the surgical site, the desired surgical instruments may be passed endoscopically through the endoscope 20 to the inflatable orthopedic device 10 for replenishment of any growth agent or modifications to the inflatable orthopedic device 10.

After the inflatable orthopedic device 10 is passed to the surgical site and placed within the bone structure, a stabilizer 40 may be passed through the incision to retain the inflatable orthopedic device 10 in a desired position. The stabilizer 40 generally maintains the inflatable orthopedic device 10 in its optimum load bearing position during removal of the endoscope 20. As depicted in FIG. 2, the stabilizer 40 is comprised of housing 42, a shaft 44, preferably threaded for rotational receipt within the housing 42, a selector, a crown 46 extending from the threaded shaft 44, a lip 50, and mechanical fasteners 54. The stabilizer's 40 components may be made of a biocompatible material such as steel, titanium, medical grade polymers.

The housing 42, illustrated in FIG. 2, is generally hollow with a cylindrically shaped threaded opening for rotational receipt of the threaded shaft 44. The housing 42 also includes a flange with the lip 50 downwardly depending therefrom. The lip 50 has apertures for the receipt of a desired number of mechanical fasteners 54, such as screws or bolts. The housing 42 is engages the incision and providing access to the growth agent containing inflatable orthopedic device 10.

As further illustrated in FIG. 2, the crown 46 is shaped for retaining the inflatable orthopedic device 10 in the desired location. As the selector is rotated, the threaded shaft extends distally towards the outer membrane 11 associated with the inflatable orthopedic device 10. The crown 46, preferably extends outwardly for engagement with the outer membrane 11 in a noninvasive manner, the crown 46 preferably having a frustoconical shape and a base secured to the shaft 44.

The threaded shaft 44 is cylindrically received by the housing 42 for telescopic receipt. As the selector is adjusted, the threaded shaft 44 moves distally or proximally along a central axis extending along the housing 42. Using the selector, the threaded shaft 44 and crown 46 retain the inflatable orthopedic device 10 in the desired location. The location of the crown 46 in associations with the inflatable orthopedic device 10 can be adjusted to influence the load bearing and load distribution of the surrounding bone surface onto the inflatable orthopedic device 10. As the shaft 44 is distally positioned, the outer membrane 11 associated with the inflatable orthopedic device 10 may be further deformed providing the desired contact profile.

In one embodiment of operation, the present invention may include providing an incision for passage of the endoscope to the surgical site associated with bone structure adapted for receiving the growth agent. The endoscopic passage 24 is guided through the incision to the surgical site, preferably the interior of the bone structure. The inflatable orthopedic device 10 may then be placed in a deflated state within the endoscopic passage 24. The distal end of the endoscopic passage 24 may then be guided to the optimum position of the surgical site. Then the growth agent delivery system 30 is engaged to the endoscopic passage 24 and growth agent is placed in the growth agent supply 34. The growth agent may be transmitted from the dispenser 33 by depressing the actuator 32 through the interior surface of the endoscopic channel 25 to the inflatable orthopedic device 10. As the growth agent transmits and inflates the inflatable orthopedic device within the bone structure. The endoscopic valve 27 is disengaged and the contents of the growth agent supply 34 are delivered to the inflatable orthopedic device 10. The stabilizer 40 may be extended from the endoscopic connector along the endoscopic passage to the inflatable surgical device 10. The endoscopic passage 24 may then be removed from the surgical site the housing 42 may be passed through the incision onto the stabilizer. The selector 48 is selectively adjusted for positioning the crown 46 associated with the distal end of the threaded shaft 44 in desired contact with the inflatable orthopedic device 10. Mechanical fasteners 54 may be used to secure the housing in place.

The preferred forms of the invention described above are to be used as illustration only, and should not be utilized in a limiting sense in interpret the scope of the present invention. Obvious modifications to the exemplary embodiments, as herein set forth, could be readily made by those skilled in the art without depart from the spirit of the present invention.

The inventor hereby states his intent to rely on the Doctrine of Equivalents to determine and assess the reasonably fair scope of the present invention as pertains to any apparatus not materially departing from but outside the literal scope of the invention as set forth in the following claims.

Having thus described the invention, what is claimed as new and desired to be secured by Letters Patent is as follows:

1. A polymer releasing orthopedic treatment system for selectively delivering a growth agent to a surgical site associated with a bone structure that was borne by endoscopically debrided osseus tissue and adapted for receiving the growth agent, the polymer releasing orthopedic treatment system comprising:
an endoscope having an endoscopic passageway extending between an endoscopic connector and the surgical site,
an inflatable orthopedic device in communication with a growth agent delivery system, adapted for delivery of said growth agent to said surgical site through said endoscopic connector,
said endoscopic passage adapted for receipt of the inflatable orthopedic device,
said inflatable orthopedic device having a receiving end and an outer membrane,
said receiving end receiving said growth agent for inflation of said outer membrane, and
said outer membrane having at least one dispersing site located along said outer membrane for selective dispersement of said growth agent to said bone structure.

2. The system according to claim 1 wherein said endoscopic connector provides access to the endoscopic passage 24 for transmitting a transport instrument through the endoscopic passage to the surgical site.

3. The system according to claim 1, wherein said growth agent delivery system further comprises:
an actuator,
a dispenser in reciprocal receipt of said actuator and adapted for receiving the growth agent, and
a dispensing valve in communication with said endoscope and adapted for transmission of the growth agent from said dispenser along said endoscopic passage.

4. The system according to claim 3, wherein said endoscope further includes an endoscopic valve extending outwardly from said endoscopic connector, said endoscopic valve in communication with said dispensing valve.

5. The system according to claim 1 further comprising a flap seal at a distal end of said endoscopic passage.

6. The system according to claim 1 further comprising a stabilizer passed through said endoscope and adapted to retain said inflatable orthopedic device.

7. The system according to claim 6 wherein said stabilizer further comprises:
a housing,
a shaft adapted for rotation within said housing; and
a crown fixed to said shaft and adapted for retaining the inflatable orthopedic device.

8. The system according to claim 7 wherein said housing further comprises a downwardly depending lip.

9. A polymer releasing orthopedic treatment system for selectively delivering a growth agent to a surgical site associated with a bone structure that was borne by endoscopically debrided osseus tissue and adapted for receiving the growth agent, the polymer releasing orthopedic treatment system comprising:
an endoscope having an endoscopic passageway extending between an endoscopic connector and the surgical site,
an inflatable orthopedic device in communication with a growth agent delivery system, adapted for delivery of said growth agent to said surgical site through said endoscopic connector,
said endoscopic passage adapted for receipt of the inflatable orthopedic device,
said inflatable orthopedic device having a receiving end and an outer membrane,
said growth agent delivery system further comprising an actuator, a dispenser in reciprocal receipt of said actuator and adapted for receiving the growth agent, and a dispensing valve,
said receiving end receiving said growth agent for inflation of said outer membrane,
said outer membrane having at least one dispersing site located along said outer membrane for selective dispersement of said growth agent to said bone structure, and
a stabilizer including a shaft rotationally receiving by a housing and a crown fixed to said shaft and adapted for retaining the inflatable orthopedic device.

10. A method for selectively releasing a growth agent that promotes bone growth within a bone structure, comprising the steps of:
providing a growth agent,
selectively delivering the growth agent along an endoscopic passageway to an inflatable orthopedic device,
inflating said inflatable orthopedic device with said growth agent,
dispensing said growth agent through said outer membrane at a dispersing site to said bone structure, and
selectively retaining said inflatable orthopedic device along said bone structure with a stabilizer by selectively adjusting a crown fixed to a shaft rotationally received by a housing.
